(19) United States
(12) Patent Application Publication
(10) Pub. No.: US 2025/0258092 A1
(43) Pub. Date: Aug. 14, 2025

(54) MULTI-GAS SENSING AND DETECTION SYSTEM

(71) Applicant: Heath Consultants Incorporated, Houston, TX (US)

(72) Inventors: John Wilkes, Cambridgeshire (GB); Euan Smith, Cambridgeshire (GB); Duncan Wales, Cambridgeshire (GB); Matthew Homburg, Cambridgeshire (GB); Joe Griffiths, Cambridgeshire (GB); Vineet Aggarwal, Richmond, TX (US)

(73) Assignee: Heath Consultants Incorporated, Houston, TX (US)

(21) Appl. No.: 19/053,210

(22) Filed: Feb. 13, 2025

Related U.S. Application Data
(60) Provisional application No. 63/552,978, filed on Feb. 13, 2024.
(60) Provisional application No. 63/552,996, filed on Feb. 13, 2024.

(51) Int. Cl. G01N 21/3504

(52) U.S. Cl. CPC G01N 21/3504; G01N 2201/0633; G01N 2201/068

(57) ABSTRACT

A non-dispersive near infrared light is used for detecting analyte gases in a testing zone. A sample obtained from the testing zone is irradiated with a reference light and with test lights. The test lights are emitted in sequence and modulated to be out of phase with the reference light. Frequencies of the test lights are in the absorption bands of particular analyte gases, with the reference light frequency being outside of the absorption bands. Analyte presence in the sample is identified by sensing changes in differences over time of test light and reference light intensity. Before irradiating the sample, the lights are optically conditioned by aligning them on the same path, and splitting the bandwidth of some lights for detecting analytes with overlapping absorption bands.

### CROSS-REFERENCE TO RELATED APPLICATION

This application claims priority from U.S. Provisional Application Ser. No. 63/552,978, filed Feb. 13^{th}, 2024, and U.S. Provisional Application Ser. No. 63/552,996 filed on Feb. 13, 2024, the full disclosures of which are incorporated by reference herein in their entireties and for all purposes.

### BACKGROUND OF THE INVENTION

### 1. Field of Invention

The present invention relates generally to a gas detection apparatus and method which utilizes the absorption of light to detect the presence of gases of interest at an extended range. The present invention uses non-dispersive infra-red (NDIR) technology.

### 2. Description of Prior Art

Different types of gases have unique light absorption characteristics. In other words, each gas type absorbs different optical frequencies. A gas analyzer system can utilize the unique absorption characteristics to identify whether a particular type of gas is present in a gas sample.

In the mid-infra-red range (approximately 2 μm-10 μm) many gases of interest have optical absorption bands. Methane (CH₄) has two strong broad absorption bands in the mid-IR spectral range centered at ˜3.3 μm and ˜7.7 μm (microns). Ethane (C₂H₆) has a strong absorption band located at 3.34 μm. Absorption bands of carbon dioxide (CO₂) are centered at 4.3, 2.7, and 2 μm. Absorption bands of carbon monoxide (CO) are centered at 2.35 and 4.66 μm.

As will be discussed below, a “gap” exists in the range of 3.8 to 4.1 μm where the light is not absorbed by the gases of interest. NDIR gas detection systems often use a reference band of 3.95 microns. Conventional gas analyzer systems typically have their own operating procedures, and calibration procedures to collect accurate data.

The Beer-Lambert law, which can be expressed as I=I₀e^{-σnl}, generally, defines a relationship that relates the absorption of a light to properties of the material irradiated by the light a gas type present in an unknown gas sample is often identified based on the Beer-Lambert law. As different materials such as gases absorb different frequencies of light energy, by passing optical energy through a gas sample and then detecting the frequencies of optical energy that are absorbed by the gas sample, the type of gas present in the sample is determinable. Further, the amount of absorption by the sample can indicate the concentration of a specific gas.

The optical transmission of a monochromatic source through a volume of absorbing gas follows the Beer-Lambert law. U.S. Pat. No. 7,835,005 discloses a device for detecting the concentration of multiple types of gases in a sample gas by absorbance measurements of the optical signal in multiple frequency bands. U.S. Pat. No. 10,545,089 discloses a technique for distinguishing methane from hydrocarbons and natural gas by using a first, second and reference wavelengths to calculate the actual concentration of methane in a gas.

### SUMMARY OF THE INVENTION

Disclosed herein is an example of a method of evaluating an environment, which includes irradiating a sample volume of the environment with light at a reference wavelength, irradiating a sample volume of the environment with light at a test wavelength, the test wavelength being a wavelength that is absorbed by an analyte gas and having an intensity that is modulated to be out of phase with an intensity of the light at the reference wavelength, sensing an intensity of the light at the reference wavelength that is transmitted through the sample volume to define a sensed reference light intensity, sensing an intensity of the light at the test wavelength that is transmitted through the sample volume to define a sensed test light intensity, comparing the sensed reference light intensity with the sensed test light intensity, and identifying the presence of the analyte gas in the environment based on the step of comparing the sensed reference light intensity with the sensed test light intensity. An example of comparing the sensed reference light intensity with the sensed test light intensity includes identifying a change over time of a difference between the sensed reference light intensity and the sensed test light intensity. In one example, the analyte gas is a first analyte gas, the light at the test wavelength is a first light, and the test wavelength is a first test wavelength, the method further includes, identifying a second analyte gas present in the sample volume of the environment by irradiating the sample volume of the environment with a second light that is at a second test wavelength, where the first light includes light generated by a first light source and the second light includes light generated by a second light source having the same characteristics as the first light source. An alternative to this example further includes forming the first light by filtering a lower portion of the bandwidth of light from the first light source, and forming the second light by filtering an upper portion of the bandwidth of light from the first light source. Further optionally, absorption bands of the first and second analyte gases overlap. In an alternative, the light at the test wavelength is a first light at the test wavelength and the test wavelength is a first test wavelength, the method further including irradiating the sample volume of the environment with a plurality of lights at a plurality of test wavelengths, each of the plurality of test wavelengths being different from the first test wavelength and being different from any of the other plurality of test wavelengths, and each of the plurality of lights having an intensity that is modulated to be out of phase with an intensity of the light at the reference wavelength. This alternative further includes controlling irradiation of the sample volume so that over a designated period of time the sample volume is irradiated with the first light at the test wavelength or one of the plurality of lights at the plurality of test wavelengths light, further options include one or more of, the sample volume being continuously irradiated with the light at the reference wavelength and each of the plurality of test wavelengths being a wavelength that is absorbed by a different analyte gas. Examples of the environment include an ambient fluid within a residential area, a landfill, a construction site, a gas distribution facility, a gas storage facility, an area where first responders have been summoned, an industrial area, a public area, any area, space or place where the presence and/or quantity of a designated substance is being sensed, detected, or measured, and combinations. In examples, the analyte gas is one or more of a noxious gas, carbon monoxide, carbon dioxide, hydrocarbon gases, methane, or ethane. The irradiating and sensing are optionally performed using a sensing system that includes a controller for identifying the analyte gas and a reference vial having a substance in a known concentration, the method further includes calibrating the sensing system by irradiating the reference cell with the reference and test lights, sensing the intensity of the test lights being transmitted through the reference cell, and adjusting the sensed test and reference light intensities transmitted through the sample volume based on sensed intensities being transmitted through the reference cell.

Also disclosed is an example of a system for evaluating an environment, which includes a reference light source emitting a reference light having a time varying intensity, a test light source emitting a test light having a time varying intensity modulated to be out of phase with the reference light and having a wavelength that is within an absorption band of an analyte gas, a space in which a sample volume of the environment is contained, an optical system comprising, an inlet in the path of the reference light, an inlet in the path of the test light, and an exit, an emission of light comprising reference light and test light, which projects from the exit and passes through the space and the sample volume of the environment, and a sensor strategically disposed in a path of the emission of light and on a side of the space opposite the exit. In one example, the sensor is responsive to an intensity of light and the system further includes a controller configured to receive signals from the sensor representing light intensity and identify the presence of an analyte gas in the sample volume. The optical system optionally includes dichroic mirrors and collimators for collimating the reference light emitted from the reference light source and the test light from the test light source onto the dichroic mirrors, and where the collimators are supported in place by the dichroic mirrors. In one example, the test light source and test light are a first test light source and a first test light, the system further including a second test light source having the same characteristics as the first test light source, the optical system including a band pass filter having an inlet in the path of the first test light and second test light and an exit from which a modified first test light is emitted having a bandwidth that is in the upper half of a bandwidth of the first test light and a modified second test light is emitted having a bandwidth that is in the lower half of a bandwidth of the second test light, where the modified first test light is in the absorption band of a first analyte gas and the modified second test light is in the absorption band of a second analyte gas, and where the absorption bands of the first and second analyte gases overlap. Embodiments exist in which the system further includes a calibration system having an arm that is selectively pivotable and a reference vial containing an analyte gas of a known concentration, where the reference vial is coupled to the arm and selectively pivoted into the path of the emission of light. The system optionally includes a housing covering other elements of the system, a power supply in the housing, and a handle selectively held by an operator when the system is transported to and used in remote environments. In alternatives, the sample volume of the environment is contained in a gas cell comprising a container selected from the group consisting of a low concentration gas cell and a high concentration gas cell. In an example, the test light source and test light are a first test light source and a first test light, the system further having a plurality of test light sources from which a plurality of test lights are selectively emitted, where the first test light and the plurality of test lights are each sequentially emitted and are modulated to be out of phase with the reference light, and where the reference light is continuously emitted.

### BRIEF DESCRIPTION OF THE DRAWINGS

Some of the features and benefits of the present invention having been stated, others will become apparent as the description proceeds when taken in conjunction with the accompanying drawings, in which:

FIG. 1 is a schematic of a system for sensing analyte gases present in an environment ambient to the system. FIG. 2 is a schematic example of operation of the system of FIG. 1.

FIGS. 2A-2D are schematic examples of the optical filtering performed by the system.

FIG. 2E is a photo of an embodiment of the source system shown in a perspective view.

FIGS. 3A-3G are graphical illustrations of examples of amplitude modulation and multiplexing of light during operation of the system of FIG. 1.

FIGS. 4 and 5 are sectional views of examples of gas cells for use with the system of FIG. 1.

FIG. 5A is a perspective view of components in the gas cell of FIG. 5.

FIGS. 6A-6E are side and overhead views of examples of the system in a non-calibrating mode and a calibrating mode.

FIGS. 7A-7C are perspective views of example embodiments of multi-gas measurement and detection tools having the system of FIG. 1.

FIG. 8 is a perspective view of an example of a head module for use with the multi-gas measurement and detection tools of FIGS. 7A-7C.

FIGS. 9A and 9B are examples of orientations of displays of the head module of FIG. 8.

FIGS. 10A-10D are examples of images displayed on the head module of FIG. 8.

While subject matter is described in connection with embodiments disclosed herein, it will be understood that the scope of the present disclosure is not limited to any particular embodiment. On the contrary, it is intended to cover all alternatives, modifications, and equivalents thereof.

### DETAILED DESCRIPTION OF INVENTION

The method and system of the present disclosure will now be described more fully hereinafter with reference to the accompanying drawings in which embodiments are shown. The method and system of the present disclosure may be in many different forms and should not be construed as limited to the illustrated embodiments set forth herein; rather, these embodiments are provided so that this disclosure will be thorough and complete, and will fully convey its scope to those skilled in the art. Like numbers refer to like elements throughout. In an embodiment, usage of the term “about” includes +/−5% of a cited magnitude. In an embodiment, the term “substantially” includes +/−5% of a cited magnitude, comparison, or description. In an embodiment, usage of the term “generally” includes +/−10% of a cited magnitude.

It is to be further understood that the scope of the present disclosure is not limited to the exact details of construction, operation, exact materials, or embodiments shown and described, as modifications and equivalents will be apparent to one skilled in the art. In the drawings and specification, there have been disclosed illustrative embodiments and, although specific terms are employed, they are used in a generic and descriptive sense only and not for the purpose of limitation.

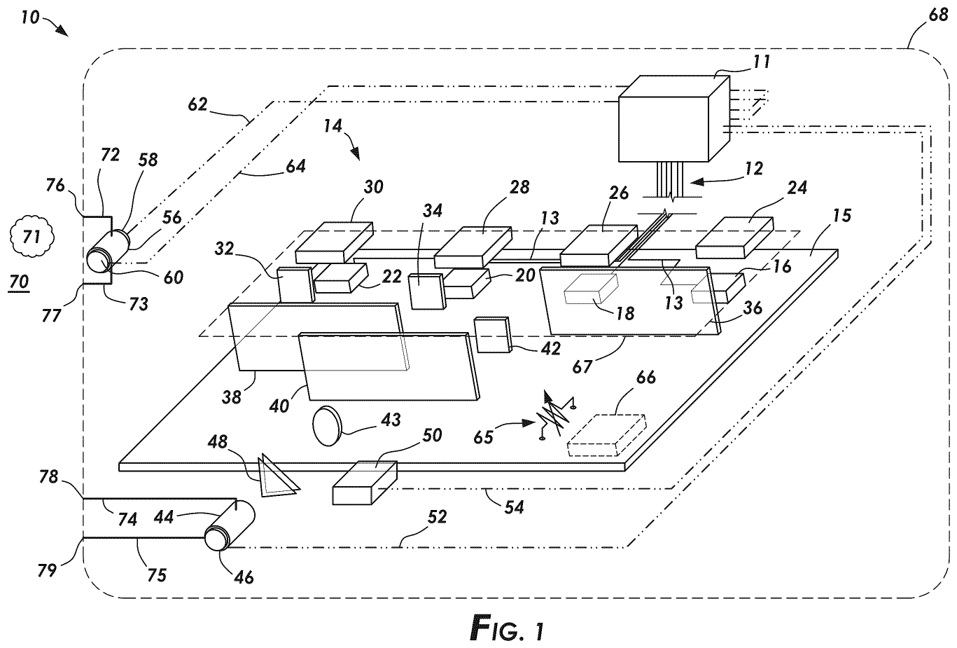

Shown in perspective view in FIG. 1 is a schematic example of a sensing system 10 for use in sensing for and detecting the presence of a fluid or fluids, such as in an example an analyte gas in a sample of gas obtained from a space or an environment ambient to the sensing system 10. A controller/power supply (“CPS”) 11 is included with the system 10, and which provides electrical power and/or control signals to components in the system 10 via a bus 12 shown connected to CPS 11. The bus 12 includes leads 13 shown extending to a source assembly 14 that includes an aluminum core printed circuit board (“PCB”) 15. The source assembly 14 includes a plurality of light emitting diodes (“LEDs”) 16, 18, 20, 22 mounted on the PCB 15 and collimators 24, 26, 28, 30 are respectively disposed adjacent to the LEDs 16, 18, 20, 22, and in an example embodiment include high precision molded elliptical mirrors. Also on the PCB 15 are band pass filters 32, 34, which as shown are generally planar and oriented substantially perpendicular to the PCB 15. In this example band pass filters 32, 34 are adjacent the collimators 30, 28, respectively. A dichroic filter 36 is shown mounted on the PCB 15 and having a length so that it has portions adjacent each of the collimators 24, 26. The dichroic filter 36 is a planar member, and similar to the band pass filters 32, 34, is oriented substantially perpendicular to the PCB 15. Another dichroic filter 38, which is also planar, is shown mounted perpendicular to the PCB 15 and spaced a distance forward of the band pass filters 32, 34 and lateral to the dichroic filter 36. An additional dichroic mirror 40 is shown spaced forward of the dichroic filter 38. The dichroic mirror 40 is a planar member oriented substantially perpendicular to the PCB 15 and mounted on the PCB 15. Another bandpass filter 42 is shown provided on the PCB 15, and perpendicular to the PCB 15 in a space laterally between the dichroic mirror 36 and dichroic mirror 40. An output lens 43 spaced forward of the dichroic mirror 40 is mounted on the PCB 15. Examples of the LEDs 16, 18, and 20 are obtainable from Hamamatsu at (hamamatsu.com), and LED 22 is obtainable from Nanoplus (Contact|nanoplus), having an optical output that is configured for the detection of carbon monoxide. In examples, the light generated by the LEDs 16, 18, 20, 22 is infrared, and having a wavelength ranging from about 0.78 microns to about 1000 microns, about 2 microns to about 50 microns, about 2 microns to about 10 microns.

The sensing system 10 of FIG. 1 further includes a low concentration gas cell 44 shown spaced adjacent the PCB 15 and strategically located to be in an optical path of the output lens 43. A photo detector 46 is shown mounted onto an end of the low concentration gas cell 44 opposite the output lens 43. An optional beam splitter 48 is illustrated between the output lens 43 and the low concentration gas cell 44. Spaced laterally from the beam splitter 48 is a reference detector 50. Communication links 52, 54 are shown respectively connected to the photo detector 46 and the reference detector 50. Communication links 52, 54 provide a means for selectively communicating signals, such as electrical and/or electromagnetic, between the controller 11 and photo detector 46 and/or between the controller 11 and the reference detector 50. A high concentration gas cell 56 is included with the sensing system 10 that is spaced laterally away from an edge of the PCB 15. The high concentration gas cell 56 is equipped with a light source 58 for emitting a light within the high concentration gas cell 56, in examples light source 58 is a micro electrical mechanical system (“MEMS”) blackbody thermal infrared source, such as that available at https://www.axetris.com. In alternatives, light source 58 is generated by one or more of the light sources described herein, and where the light generated is processed by one of the modulation schemes described below. In the illustrated example, the high concentration gas cell 56 also includes a photo detector 60 that selectively senses intensity of light emitted from the light source 58, which reaches the end of the high concentration gas cell 56 opposite from the light source 58. Embodiments of the photo detector 60 include a four channel pyroelectric detector, examples of which are available from Infratec at https://www.infratec.co.uk/and having channels for the detection of CH₄ (3.33 μm), CO₂, H₂O, and a reference channel. Inside the high concentration gas cell 56 is an ellipsoidal reflector with the light source 58 and detector 60 placed at the foci. Communication links 62, 64 are shown providing communication between the controller/power source 11 and the light source 58 and photo detector 60 respectively. Examples of the communication links 52, 54, 62, 64 include conductive materials, fiberoptics, means for transmitting and receiving wireless signals, and combinations.

Still referring to FIG. 1, a thermistor 65 is shown mounted on the PCB 15 for monitoring the temperature of the PCB 15. The thermistor 65 is selectively in communication with a thermal electric cooler 66 shown in dashed outline and mounted on a lower surface of the PCB 15. In one example, the thermoelectric cooler 66 is a Peltier or Peltier-type cooler. Also shown in dashed outline is a support plate 67 disposed upwards from the PCB 15 on upper lateral edges of the filters 32, 34, 42 and/or the dichroic mirrors 36, 38, 40. Further in this example, the collimators 24, 26, 28, 30 are secured onto the support plate 67 and suspended in precise locations and orientations with respect to the LEDs 16, 18, 20, 22. An advantage of this arrangement is that the source assembly 14 is a thermally stable unit, and its performance and output accuracy is not degraded when subjected to expected operating temperatures, as in examples, members supporting the plate 67 above the board 15, i.e., one or more of filters 32, 34, 42 and mirrors 36, 38, 40 are formed from a silicon based material that does not expand or contract in size by an amount that affects or disturbs a precise alignment between the LEDs 16, 18, 20, 22 and the collimators 24, 26, 28, 30. Advantages also exist by the use of silicone and other materials having a high coefficient of thermal conductivity, which quickly transfers heat so that thermal effects are not experienced due to disparate thermal expansion in different components.

A housing 68, illustrated in dashed outline in FIG. 1, surrounds the controller/power supply 11, gas cells 44, 56, and source assembly 14 separating these components from ambient fluid 70 that makes up the environment surrounding the sensing system 10. In an embodiment, a sample volume, such as the ambient fluid 70, is obtained from the environment, which in alternatives is obtained using the sensing system 10 or any other sampling device. In examples, the sensing system 10 is a handheld portable device, manually operated by an individual for testing the ambient fluid 70 for a designated substance(s), such as an analyte gas 71. Testing the ambient fluid 70 includes investigating, sensing, detecting, and/or identifying the presence and quantity of the designated substance in the ambient fluid 70. Examples of the ambient fluid 70 include ambient air in a particular space within an area, such as a residential area, a landfill, construction site, gas distribution facility, gas storage facility, an area where first responders have been summoned, an industrial area, a public area, or at any area, space or place where the presence and/or quantity of a designated substance is being sensed, detected, or measured. Specific examples of analyte gas 71 include noxious gases, such as carbon monoxide, carbon dioxide, and hydrocarbon gases, including but not limited to methane and ethane. In a non-limiting example of use, an amount of ambient fluid 70 from ambient is drawn into the housing 68 through a line 72 where it is directed to the high concentration gas cell 56 and discharged from the high concentration gas cell 56 through a line 73. Similarly, ambient fluid 70 to be analyzed in the low concentration gas cell 44 is drawn to the low concentration gas cell 44 through a line 74 and discharged from the low concentration gas cell 44 through a line 75. Openings 76, 77, 78, 79 penetrate through the housing 68 to provide communication between ambient and lines 72, 73, 74, 75.

Examples of operation of the sensing system 10 include manually transporting the system 10 to a particular space where ambient fluid 70 is to be tested. The ambient fluid 70 is drawn into the cells 44, 56 where it is illuminated with light. Light from the light source 58 irradiates the fluid 70 in the high concentration gas cell 56, and as explained in more detail below, light from the LEDS 16, 18, 20, 22 is captured, focused, imaged, and filtered through the components in the system 10, and then directed to the high concentration cell 56 where it illuminates the fluid 70 in the low concentration gas cell 44. The presence and amount of the analyte gas 71 in the cells 44, 56 is estimated by measuring an amount of optical absorption of light emitted into the cells 44, 56. In an example, a particular analyte is identified inside the cells 44, 56 by emitting light into the cell(s) 44, 56 having a wavelength that is within an optical absorption band of the particular analyte, and an absorption of that light is detected within the cell(s) 44, 56. Examples of detecting absorption include comparing an intensity of light emitted into the cells 44, 56 with an intensity of light sensed by the photodetectors 46, 60. Optionally, software in or accessed by controller/power supply 11 provides instructions for this comparison. In one example of operation, the LEDs 16, 18, 20, 22 are energized in a time modulated fashion so that a magnitude of light output from one of the LEDs 16, 18, 20, 22 is out of phase with the other LEDs 16, 18, 20, 22. The light output that is out of phase is referred to as a reference light, and lights from the LEDs that are in phase with one another and out of phase with the reference light are referred to as test lights. Embodiments exist in which the phase difference between the reference light and each of the test lights ranges up to about 180°. In an alternative, the reference light is at a wavelength outside optical absorption bands of analyte gases to be detected, and test light wavelengths are within optical absorption bands of analyte gases to be detected. As described in more detail below, the reference light and one or more of test lights simultaneously irradiate the low concentration gas cell 44, and the intensity of the reference and test lights are measured by the photodetector 46. In examples in which none of the test lights is absorbed by gas in the low concentration gas cell 44 (and thus the wavelengths of the test lights are all outside of the optical band of gas in the low concentration gas cell 44), because the intensity of the reference and test lights are time varying and out of phase, the photodetector 46 generates an output signal that is substantially constant. In examples in which one or more of the test lights is absorbed by gas in the low concentration gas cell 44 (e.g., one or more of the wavelengths of the test lights are within an optical band of a gas in the low concentration gas cell 44), that output signal from photodetector 46 is not constant over time, but changes when the test light is absorbed by one of the gases inside the low concentration gas cell 44.

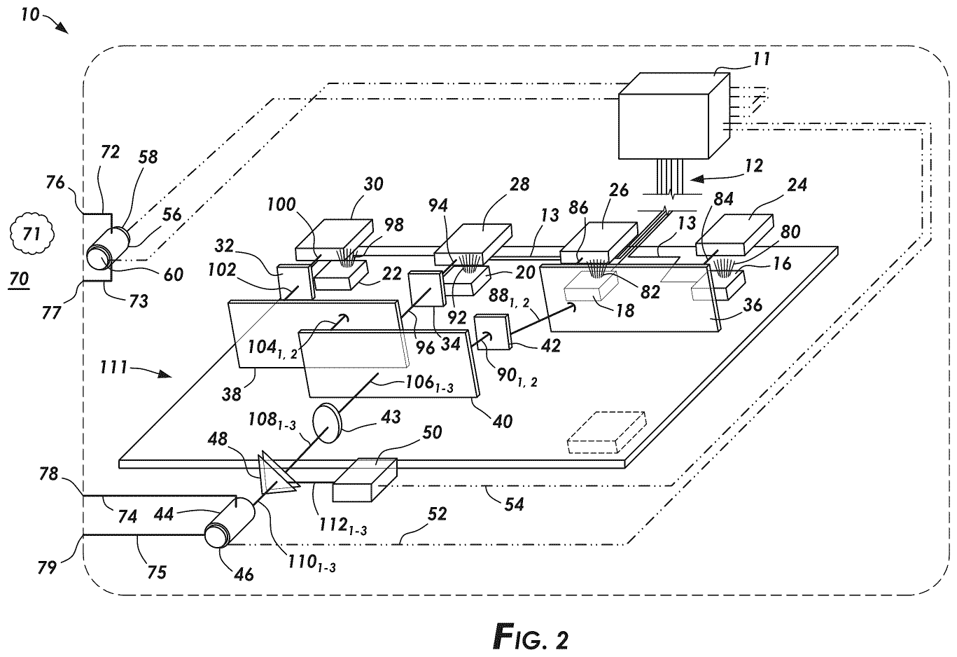

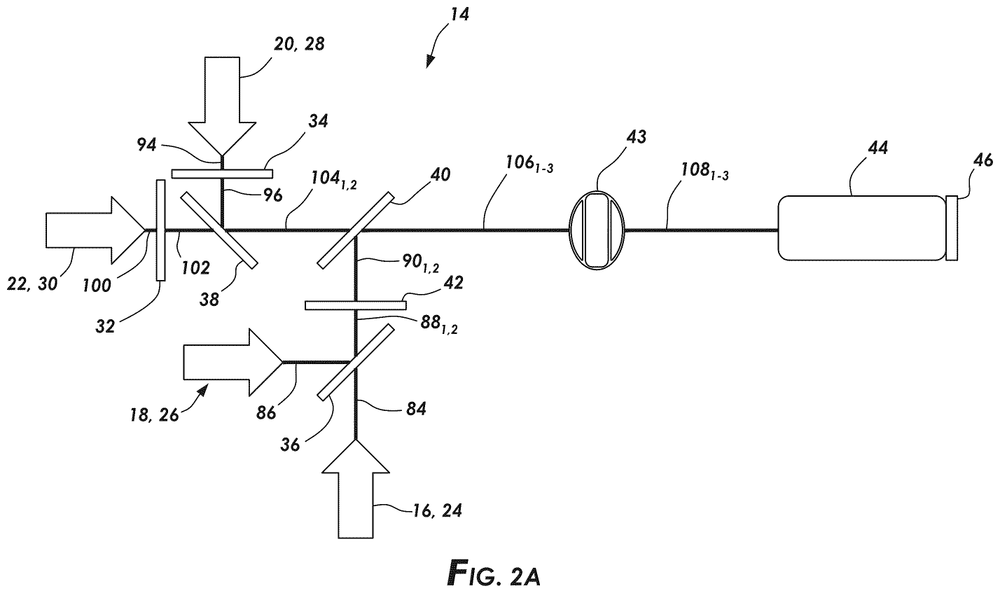

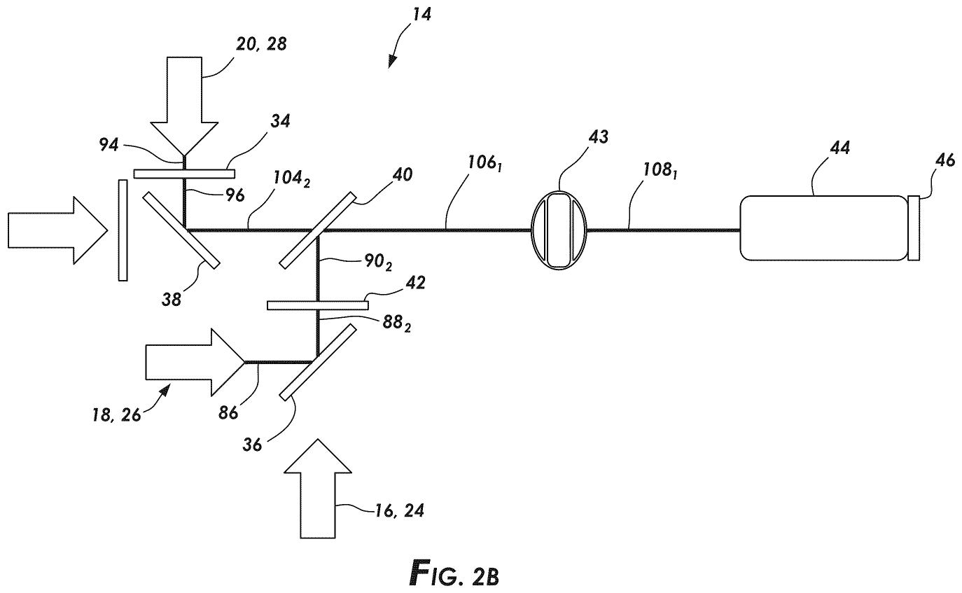

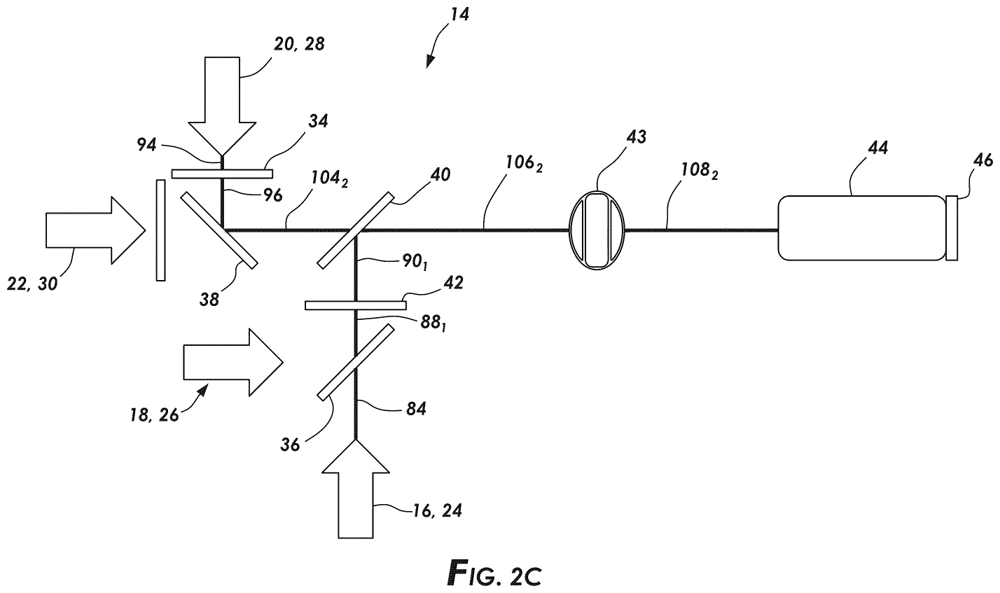

Referring now to FIG. 2, a nonlimiting example of use of the sensing system 10 is shown in which the LEDs 16, 18, 20, 22 are selectively energized to emit light, which is optically filtered for spectroscopic analysis of the ambient fluid 70 within the low concentration gas cell 44. In this example, energizing LED 16 generates light 80, a portion of light 80 is focused by the collimator 24 into focused light 84, which is directed towards dichroic filter 36. Similarly, energizing LED 18 generates light 82, and a portion of light 82 is redirected and focused by collimator 26 to create focused light 86, which is directed towards dichroic filter 36. Focused light 84 passes through and is filtered by dichroic filter 36 to create light 88₁ and focused light 86 is reflected by and filtered by dichroic filter 36 to create light 88₂, light 88_{1,2} is shown being directed towards the bandpass filter 42. Light 88_{1,2} is filtered by bandpass filter 42, which exits as light 90_{1,2} and directed towards dichroic mirror 40. The bandwidths of light 90_{1,2} are narrower than light 88_{1,2}, having been narrowed by bandpass filter 42.

Energizing LED 20 creates light 92 shown directed towards collimator 28, where it is refocused and reimaged as light 94 shown being directed towards the bandpass filter 34, and light 96 exiting the opposite side of bandpass filter 34 is directed towards dichroic mirror 38. Energizing LED 22 generates light 98 shown being directed towards collimator 30, which is reimaged and refocused as light 100 that is directed to the bandpass filter 32. Light 100 is filtered by bandpass filter 32 and exits the opposing surface of bandpass filter 32 as light 102 that is directed to dichroic mirror 38. Light 102 passes through and is filtered by dichroic filter 38 to form light 104₁ shown exiting a side of dichroic filter 38 opposite bandpass filter 32 and being directed to dichroic mirror 40. Light 96 is reflected and filtered by dichroic filter 38 and exits filter 38 as light 104₂ being directed towards dichroic filter 40. In this example, light 104₂ is the reference light, which is emitted simultaneously with each light 90₁, light 90₂, and light 104₁, whereas light 90₁, light 90₂, and light 104₁ are emitted sequentially and not simultaneous to one another. Further in this example, light 104₁ and light 104₂ are combined and transmitted through dichroic filter 40 to create light 106₁, light 104₂ and light 90₁ are combined and reflected by dichroic filter 40 to create light 106₂, and light 104₂ and light 90₂ are combined and reflected by dichroic filter 40 to create light 106₃. Each of light 106₁₋₃ are shown extending from a surface of dichroic mirror 40 towards output lens 43. Light 108₁₋₃ represents light 106₁₋₃ being refocused by lens 43 which intersects the beam splitter 48 and exits as light 110₁₋₃, which enters the low concentration gas cell 44. Collimators 24, 26, 28, 30, dichroic mirrors 36, 38, 40, band pass filters 38, 42, and lens 43 define an optical system 111. A portion of light 108₁₋₃ is redirected by the beam splitter 48 as light 112₁₋₃ to the reference detector 50. In an example, the amount of light 112₁₋₃ is a small percentage of light 108₁₋₃, which optionally ranges up to around 3% of light 108₁₋₃. The light 112₁₋₃ received by the reference detector 50 generates signals which are sent via the communication link 54 to the controller 11, and analyzed to confirm a stable and proper output from the LEDs 16, 18, 20, 22, this provides information to account for any drift from a baseline signal. An advantage of the beam splitter 48 and reference detector 50 is to provide a constant output from the LEDs 16, 18, 20, 22 and stabilize the output of any of the nondispersive infrared sources. In an alternative the reference detector 50 samples light 112₁₋₃ at about every 50 microseconds.

Schematically illustrated in FIG. 2A is an example of the optical filtering of the light generated by the LEDs 16, 18, 20, 22. In this example, LED 20 is a reference light source and produces a reference light and LEDs 16, 18, 22 are test light sources that each generate a test light, in alternatives, each test light is in a wavelength of interest to identify analyte gas 71. As described in more detail below, LEDs 16, 18, and 22 are multiplexed, i.e., each is operated at specific sequences of time while at the same the other two are not operating and not generating a test light. The LED 20 operates simultaneously with each sequence of test light generation by LEDs 16, 28, 22 so that the reference light is generated coincident with and during generation of each of the test lights. An advantage of this operation is that comparing a signal representing characteristics of each test light generated by one of LEDs 16, 18, 22 with a signal representing characteristics of the reference light generated by LED 20, the presence of an analyte gas 71 within the ambient fluid 70 is detectable. In an embodiment, the signals being compared are output signals from the photodetector 46 and/or detector 60. In this example, LED 16 and LED 18 have substantially the same construction and specifications so that lights 80, 82 (FIG. 2) emitted respectively from LEDs 16, 18 have substantially the same signatures and the light generated by and emitted from these LEDs 16, 18 have substantially the same characteristics, such as wavelength, a spatial profile, and an angular profile. Dichroic filter 36 is configured so that light 84 from collimator 24 passes through and is filtered by filter 36 to form light 88₁. Light 86 from collimator 26 is filtered by and reflected from dichroic filter 36 to create light 88₂. As light 84 and light 86 have substantially the same characteristics, the filtering by dichroic filter 36 modifies light 84 and light 86 and results in a modified light 88₂ having a bandwidth that is on a lower half of the bandwidth of light 86, and conversely results in a modified light 88₁ having a bandwidth that is on an upper half of the bandwidth of light 84. In this example, LEDs 16 and 18 are energized to operate sequentially and without their respective periods of operation overlapping, light 84 and light 86 reach filter 36 along different time periods so that light 88₁ and light 88₂ are not present at the same time. Downstream of dichroic filter 36, bandpass filter 42 receives light 88₁ and filters and modifies light 88₁ to form a narrower bandwidth light 90₁. In an alternative, a pair of bandpass filters (not shown) are upstream of dichroic filter 36 instead of the single bandpass filter 42 being downstream of dichroic filter 36. Similarly, light 88₂ is further modified by passing through bandpass filter 42 to form light 90₂ which is at a smaller bandwidth than light 88₂. Light 90₁ and light 90₂, which are shown collectively as 90₁,2 are sequentially directed towards dichroic mirror 40 that is angled with respect to the direction of light 90₁ and light 90₂.

Still referring to FIG. 2A, light 100 being emitted from LED 22 and collimator 30 passes through bandpass filter 32 to form light 102, which has a smaller bandwidth than light 100. Similarly, light 94 being emitted from LED 20 and collimator 28 passes through bandpass filter 34 to form light 96 that has a smaller bandwidth than light 94. Bandpass filters 32, 34, and 42 of FIG. 2A are configured to remove light outside of a designated bandwidth, an example of a designated bandwidth includes an absorption band of an analyte gas. An advantage of narrowing a bandwidth of light to a designated bandwidth avoids cross-talk in a sensor, such as a photodetector, when detecting the light. Dichroic filter 38 is angled so that light 102 is transmitted through dichroic filter 38 to form light 104₁ shown being directed towards dichroic filter 40. Dichroic filter 38 is also configured so that light 96 exiting bandpass filter 34 is reflected from dichroic filter 38 to form light 104₂ also shown being directed towards dichroic filter 40. In this example, LED 20 is operated substantially continuously to create the reference signal discussed above and LEDs 16, 18, 22 are energized sequentially and not simultaneously to generate test signals. As described above, collimators 24, 26, 28, 30 collimate light 80, 82, 92, 98 to respectively form light 84, 86, 94, 100; and the light 80, 82, 92, 98 is generated respectively by LEDs 16, 18, 20, 22, where LED 20 operates continuously, while LEDs 16, 18, 22 do not operate simultaneously, but instead operate sequentially in a first, second, and third time period or sequence.

Shown schematically in FIGS. 2B-2D is a non-limiting example of operation of the source tree 14 during the first, second, and third respective time periods or sequences and the light being generated and processed during these sequences. During each of the first, second, and third sequences, LED 20 operates and generates light 92 (FIG. 2). Light 92 passes through the collimator 28 to form light 94 that transmits through bandpass filter 34 to form light 96, which reflects from dichroic mirror 38 to form light 104₂, Light 104₂ is directed from dichroic mirror 38 to a side of dichroic mirror 40.

In FIG. 2B, shown is a schematic example of the first time period or sequence, during which LED 18 operates to generate light 82 (FIG. 2) while LEDs 16 and 22 are not operating. Light 86 from collimator 26 reflects from dichroic mirror 36 to form light 88₂, which is processed by bandpass filter 42 into a narrower bandwidth light 90₂ and directed to a side of dichroic mirror 40 opposite light 104₂. Light 90₂ and light 104₂ are combined by dichroic mirror 40 into light 106₁ shown directed to an upstream side of lens 43. Light 106₁ is focused by lens 43 into light 108₁ and transmitted from a downstream side of lens 43 to gas cell 44. Light 106₁ passes through gas cell 44 where it is detected by photodetector 46, which emits a signal representative of the intensity of light 108₁ sensed by the photodetector 46.

An example of the second time period or sequence is shown schematically in FIG. 2C, during the second time period LED 16 operates to generate light 80 (FIG. 2), and LEDs 18 and 22 are not operating. Light 80 is collimated by collimator 24 into light 84 that is directed to dichroic mirror 36, and exits dichroic mirror 36 as light 88₁, which is processed by bandpass filter 42 into a narrower bandwidth light 90₁ and directed to a side of dichroic mirror 40 opposite that receiving light 104₂. Light 90₁ and light 104₂ are combined by dichroic mirror 40 into light 106₂, which similar to light 106₁ is directed to an upstream side of lens 43. Light 106₂ is focused by lens 43 into light 108₂ that is transmitted from a downstream side of lens 43 to gas cell 44. Light 108₂ passes through gas cell 44 where it is detected by photodetector 46, which emits a signal representative of the intensity of light 108₂ sensed by the photodetector 46. An example of the third time period or sequence is shown in FIG. 2D, during the third sequence LED 22 operates to generate light 98 (FIG. 2), and LEDs 16 and 18 are not operating. Light 98 is collimated by collimator 30 into light 100 that is directed towards and passes through bandpass filter 32 to form light 102 having a bandwidth narrower than that of light 100. Light 102 is directed through dichroic mirror 38 and exits as light 104₁ and is directed to the same side of dichroic mirror 40 that receives light 104₂. Light 104₁ and light 104₂ are combined by dichroic mirror 40 into light 106₃ shown directed to an upstream side of lens 43. Light 106₃ is focused by lens 43 into light 108₃ and transmitted from a downstream side of lens 43 to gas cell 44. Light 108₃ passes through gas cell 44 where it is detected by photodetector 46, which emits a signal representative of the intensity of light 108₃ sensed by the photodetector 46.

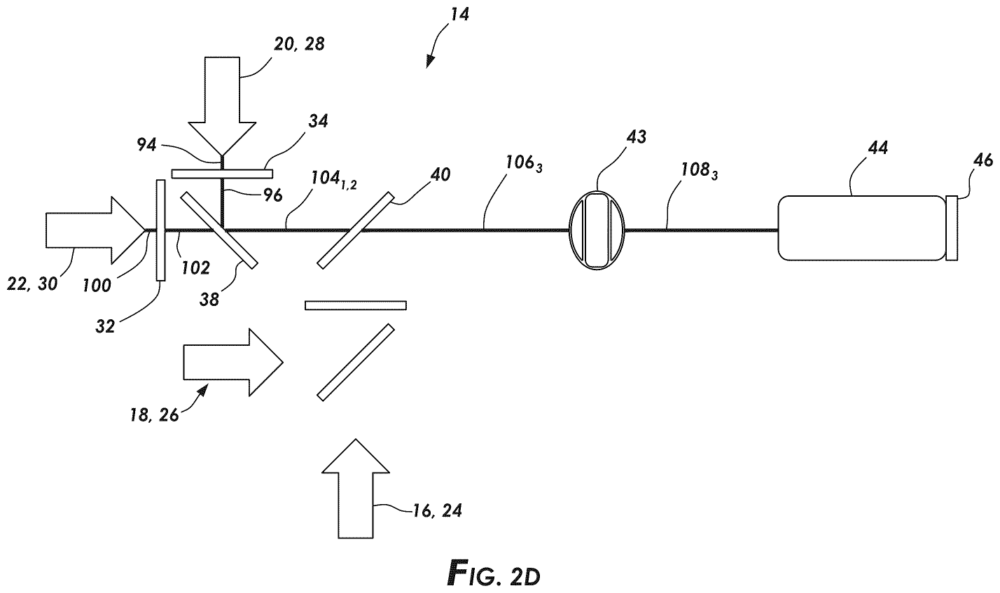

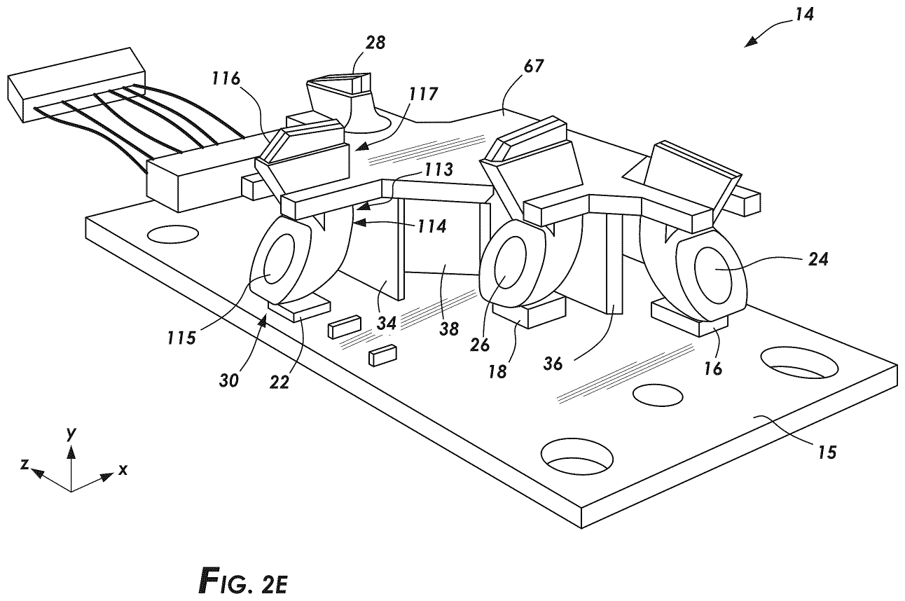

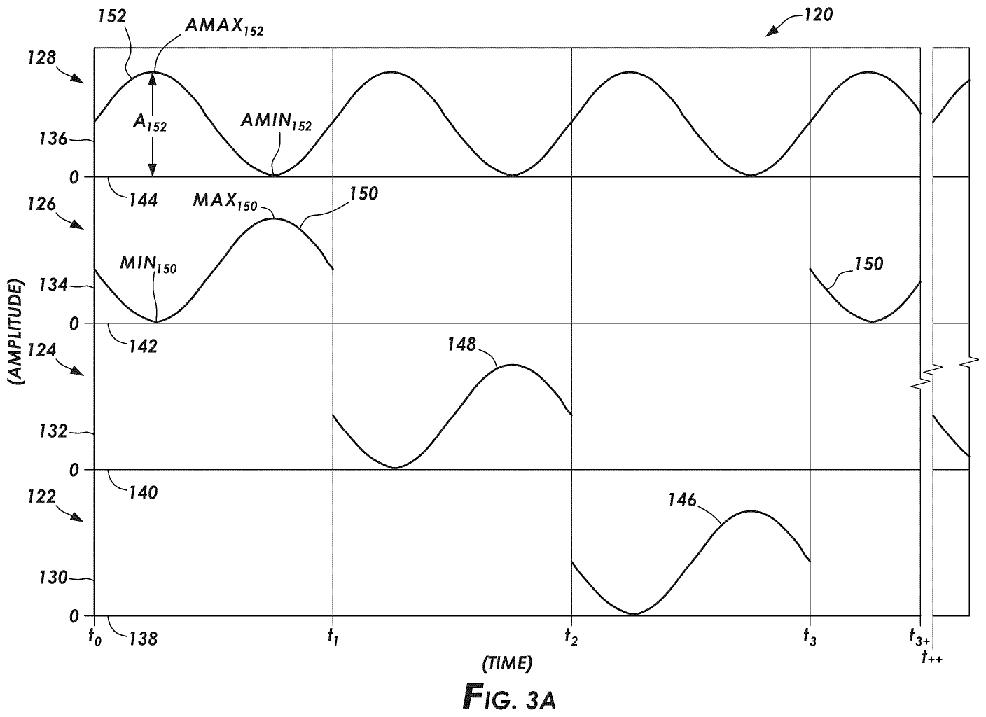

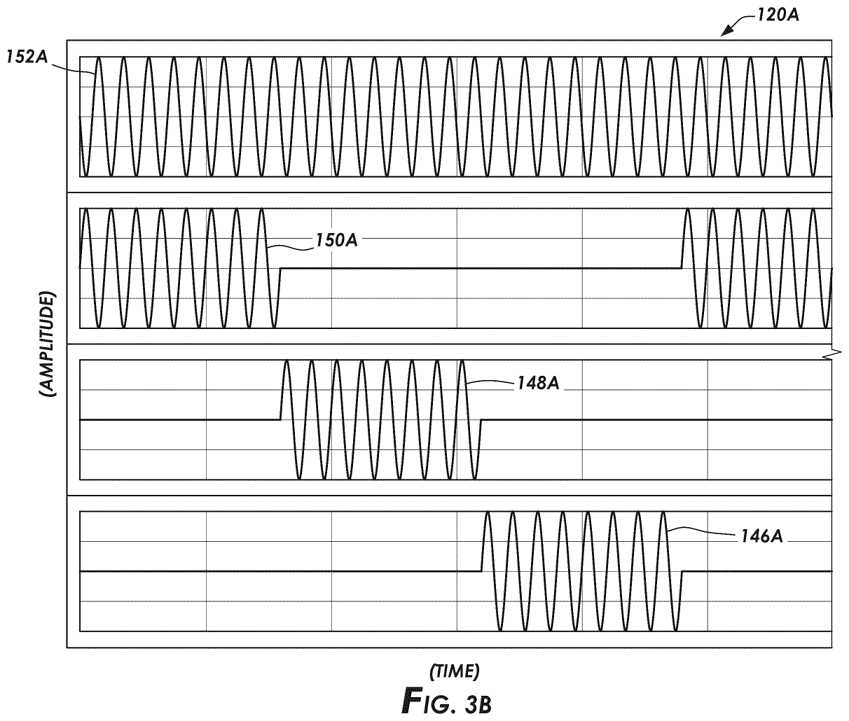

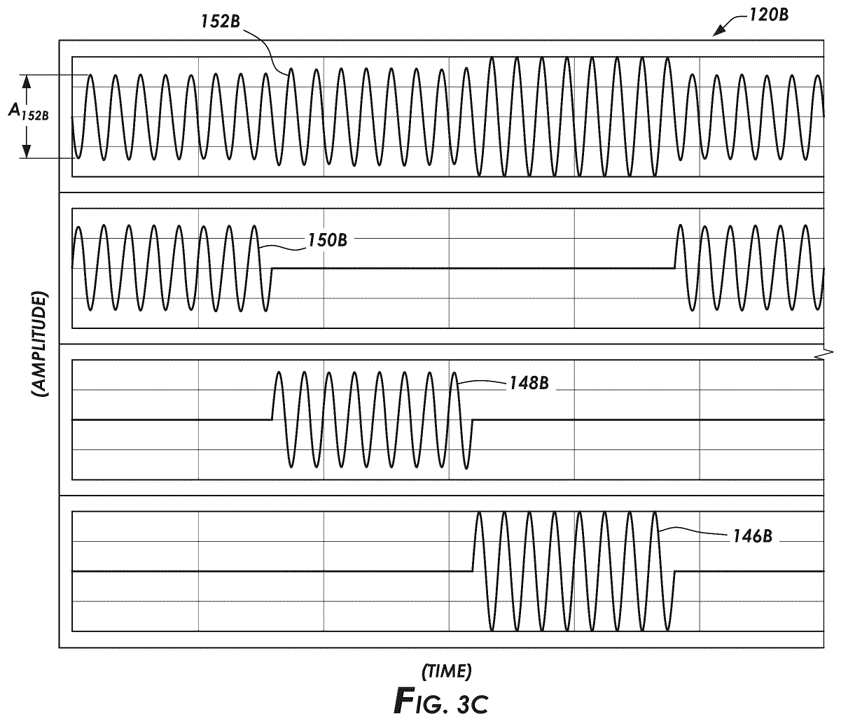

As described further below and illustrated in FIGS. 3A-3G, during each of the sequences output signals from the photodetector 46 are monitored, and when the output signal is substantially constant (alternatively referred to herein as “direct current”), it is determined there is no analyte gas present in the gas cell 44 having an absorption band coincident with a test signal being generated, transmitted, or processed during the particular sequence. Conversely, if during a particular time period or sequence, the output signal varies over time (alternatively referred to herein as “alternating current”), it is determined there is an analyte gas present in the gas cell 44 having an absorption band coincident with the test signal being generated, transmitted, or processed during the particular sequence.

An optional embodiment of the source system 14 is shown in a perspective view in FIG. 2E, a reference coordinate system is included having axes in the X, Y, and Z directions. In the embodiment shown, collimator 30 is made up of a body 113 having an ellipsoid section 114 with a major axis generally aligned with the Y axis and a minor axis generally aligned with the Z axis. A face 115 is defined where a portion of outer surface ellipsoid section 114 is generally planar, face 115 has a generally elliptical outer perimeter with a minor axis generally aligned with the Z axis and a major axis oblique to the Y axis. Body 113 includes a neck section 116 that is integrally formed onto an end of ellipsoid section 114 opposite inlet aperture. Neck section 116 has a generally rectangular cross section, and a length and height generally aligned with the X and Y axes respectively, and a width oblique with the Z axis. Neck section 116 inserts into a slot 117 formed in support plate 67, slot 117 has a length, height, and width generally aligned with that of neck portion 116, and the length and width of slot 117 are dimensioned to receive and secure neck portion 116 within so that collimator 30 is supported a designated distance from board 15 and LED 22, and maintained in position so that an inlet aperture (not shown) formed on a lower end of body registers with light 98 (FIG. 2) emitted from LED 22. A gold coated ellipsoidal mirror (not shown) is molded within ellipsoid section 114 of body 113. Collimated light exits collimator 30 through an outlet aperture (not shown) formed through an outer surface of body 113 opposite face 115. In the illustrated embodiment, while the orientation and/or location of collimators 24, 26, 28, 30 can vary, their shape, construction, and operation are the same or substantially the same as collimators. Accordingly, the details of collimator 30 provided above are applicable to that of collimators 24, 26, 28. Adhesive is optionally applied to secure the collimators 24, 26, 28, 30 to the support plate 67. Collimators 26, 30 are oriented so that light being focused by and directed from collimator 26 is substantially parallel to light being focused by and directed from collimator 30, but perpendicular to light being focused by and directed from collimators 24, 28; and light being focused by and directed from collimators 24, 28 projects parallel and in opposite directions. Also shown is that the collimators 24, 26, 28, 30 are spaced outside of the collection of the dichroic filters 36, 38 and the band pass filter 34. Further illustrated is that the dichroic filters 36, 38 and band pass filter 34 are generally planar members disposed on an upper surface of PCB 15 and perpendicular to PCB 15, and support plate 67, which is substantially parallel to PCB 15, is set on edges of dichroic filters 36, 38 and band pass filter 34 distal from PCB 15. As shown, the structure of dichroic filters 36, 38 and band pass filter 34 provide support on which the support plate 67 mounts to the PCB 15.

Graphically illustrated in FIGS. 3A-3F are examples of amplitude modulation and multiplexing, or sequencing (alternatively referred to herein as a modulation scheme), of the light and light sources discussed above. Included in FIG. 3A is a chart 120 which is made up of plots 122, 124, 126, 128 which each have a respective ordinant 130, 132, 134, 136 and an abscissa 138, 140, 142, 144. Each ordinant 130, 132, 134, 136 represents an amplitude of the intensity or power of each light 84, 86, 94, 100. Included with each of the plots 122, 124, 126, 128 are graphs 146, 148, 150, 152 illustrating an example of time varying magnitudes of electrical power provided to the LEDs 16, 18, 20, 22 for generating lights 84, 86, 94, 100. Graph 152 represents the output signal of light 92 generated by energizing LED 20, which is shown having a continuous periodic wave form and with a time varying amplitude A₁₅₂, but with repeating maximum amplitude AMAX₁₅₂ and minimum amplitude AMIN₁₅₂. In the example shown, the wave form of graph 152 generally follows a sine wave function, and having a maximum amplitude substantially equal to maximum amplitudes of each of graphs 146, 148, 150, so that in this example the maximum intensities of lights generated by LEDs 16, 18, 20, 22 are all substantially equal. Optionally, the LEDs 16, 18, 20, 22 are operated to follow other forms of repeating periodic waveforms, including but not limited to, square waves, triangular waves, complex waves, asymmetric waves, and combinations. Graphs 146, 148, 150 as shown also have periodic wave forms that are each 180° out of phase with the graph 152 of the reference signal. The wave forms of graphs 146, 148, 150 reflect the sequential operation of LEDs 22, 16, 18, respectively, and as such are not continuous, but have portions that are time varying and portions where the amplitude is constant over multiple time spans. More specifically, graph 146 is time varying over time period t₂-t₃, graph 148 is time varying over time period t₁-t₂, and graph 150 is time varying over time period tₒ-t₁ and time period t₃-t₃₊, but these graphs 146, 148, 150 are otherwise constant over time with amplitudes equal to their corresponding ordinates 138, 140, 142. In a nonlimiting example, each of light 90₁, 90₂, 104₁ is at a wavelength within the optical absorption band of an analyte 71 (FIG. 1) of interest within the sample of ambient gas 70 in the low concentration gas cell 44, in which the analytes of interest are methane, ethane, and carbon monoxide. In this example, the wavelength of light 90₂ ranges from about 3160 nm to about 3285 nm with a center at about 3227 nm—which is within the optical absorption band of methane, the wavelength of light 90₁ ranges from about 3285 nm to about 3410 nm with a center at about 3322 nm, which is within the optical absorption band of ethane, the wavelength of light 104₁ is within the optical absorption band of carbon monoxide, and the wavelength of light 104₂ is outside the optical absorption bands of the analytes of interest. Further in this example, the time varying modulation of the LEDs 16, 18, 20, 22 results in the intensity of light 104₂ varying in magnitude as illustrated by graph 152, similarly, the intensity of light 104₁ also varies in magnitude as illustrated by graph 146 over time period tₒ-t₁ and 180° out of phase with graph 152. As such, in examples in which an output of a signal from the photodetector 46 is substantially constant over time period tₒ-t₁ or t₃-t₃₊ indicates that gases having an absorption band corresponding to the wavelength light 104₁ are not present in the low concentration gas cell 44; in contrast, when the photodetector 46 signal output changes over that time period, indicates a portion of light 104₁ was absorbed by a gas in the low concentration gas cell 44, and based on the respective bands of the optical absorption band of carbon monoxide and the wavelength of light 104₁, it is deduced that the analyte fluid 70 includes carbon monoxide. Similarly, amounts of methane being present in the low concentration gas cell 44 is deduced by noticing a change in magnitude of the photodetector 46 signal over time periods t₁-t₂ when gas cell 44 is being irradiated by light 106₂, and amounts of ethane being present in the gas cell 44 is deduced by noticing a change in magnitude of the photodetector 46 signal over time periods t₂-t₃ when cell 44 is being irradiated by light 106₃. In an embodiment, the frequency of the modulation is 1 kilohertz and the span of each of the time periods t₁, t₂, t₃, etc. is 1 millisecond. An unexpected advantage realized by the placement and orientation of dichroic mirror 36 and bandpass filter 42 is the ability to generate light 90₁ and light 90₂ that when combined with light 104₂, and directed into the low concentration gas cell 44, provides resulting output signals from the photodetector 46 that distinguish the amount and presence of methane from the amount and presence of ethane in the sample of ambient fluid 71 in the low concentration gas cell 44. Sequencing light from LEDs 16, 18, 22 provides an advantage of simultaneously detecting multiple gases in cell 44, 56, and identifying if one of more of those gases 71 are present in the ambient fluid 70 and therefore in the environment.

Graphically shown in chart 120A of FIG. 3B is an example of operation similar to that of FIG. 3A, e.g., the drive scheme steps through three drive periods, one for each test channel, and then repeats. Within one drive period, each target channel LED 16, 18, 22 is amplitude modulated, with the reference LED 20 modulated with the opposite signal (180 degrees out of phase). In examples of drive schemes, the reference LED 20 is modulated so that without any optical absorption of light generated by LEDs 16, 18, 22 in gas cell 44, 56, an output at a detector 46, 60 is substantially direct current, i.e., has a substantially constant output over time because the differences between detected intensities of the reference light and test lights are constant. An alternative mode of operation is illustrated in FIGS. 3C and 3D in which an amplitude A152B of graph 152B and an amplitude A152C of graph 152C changes over time (by varying electrical power supplied to LED 20) to be equal to or substantially the same as amplitudes of graphs 146B, 148B, 150B during their respective periods of operation. An advantage of modulating electrical power supplied to the reference LED 20 provides a way of accounting for differences in power output of LEDs 16, 18, 22. FIG. 3D, like FIG. 3A, illustrates an example of modulating over a single period for each test signal. Demonstrated in FIGS. 3E and 3F are ways to modulate multiple LEDs simultaneously and detect the results. The scheme shown in FIG. 3E is referred to herein as a quadrature drive scheme, and in which LEDs 16 and 18 are time modulated to be 900 out of phase (as illustrated by the 90° offset between graphs 148C and 150C). In the chart 120E depicted in FIG. 3F, modulation occurs at different frequencies, e.g., LED 16 and 18 (for detecting methane and ethane respectively) are modulated approximately 90° out of phase with one another (graphs 148E, 150E), and at twice the frequency of LED 22 (graph 146E). In further alternatives, outputs from LEDs 16, 18, 20, 22 are modulated at frequencies to minimize the possibility of crosstalk due to nonlinearities in the system producing higher harmonic oscillations of a base band. Referring to FIG. 3G, an alternative example of operation is illustrated in chart 120F that is based on sets of digital vectors which can be used as a set of basis functions to multiplex a set of digitally modulated signals, for example Walsh functions (https://en.wikipedia. org/wiki/Walsh_function). Graphs 146F, 148F, 150F show the result of using the three non-DC vectors of an order 4 Hadamard matrix (https://en.wikipedia.org/wiki/Hadamard matrix) to drive the three test LEDs 22, 18, 16 respectively. Other Walsh functions will also work, for example the Hadamard-Paley matrix (https://en.wikipedia.org/wiki/Paley construction).

Referring now to FIG. 4, shown in a side section view is an example of the high concentration gas cell 56 with the light source 58 on one end. In this example, light 154 is emitted from the light source 58 into an elongated chamber inside the gas cell 56, which includes a sample of ambient liquid 71. The light 154 is shown reflecting from an inner surface 156 of the high concentration gas cell 56. The inner surface 156 of the gas cell 56 is elliptical and configured so that the light 154 reflecting from surface 156 is directed to the photo detector 60 at the opposing end of the gas cell 56. Sampled amounts of the ambient fluid 70 enter gas cell 56 through an inlet 72 and exit the cell 56 through an outlet 73 formed radially through sidewalls of the gas cell 56, so that in cell 56 and cell 44 (FIG. 2) is a space in which a sample volume of the ambient environment is contained. An advantage of including low and high concentration gas cells 44, 56 in the system 10 (FIG. 1) enables detection of a particular analyte gas with the low concentration gas cell 44 when the analyte gas is more dilute in the ambient fluid 70 and at a location distal from a source of the analyte gas, and when proximate the source of the analyte gas, the analyte gas is monitored using output from the high concentration gas cell 56. Alternatives exist in which output signals from detectors 46, 60 are monitored simultaneously, sequentially, or periodically. Further advantages of a system 10 that includes low and high concentration gas cells 44, 56 are extended range, compensation for cross-sensitivity to moisture, and that the system 10 avoids, or corrects, incorrect sensing due to encountering two or more analyte fluids with overlapping optical absorption bands.

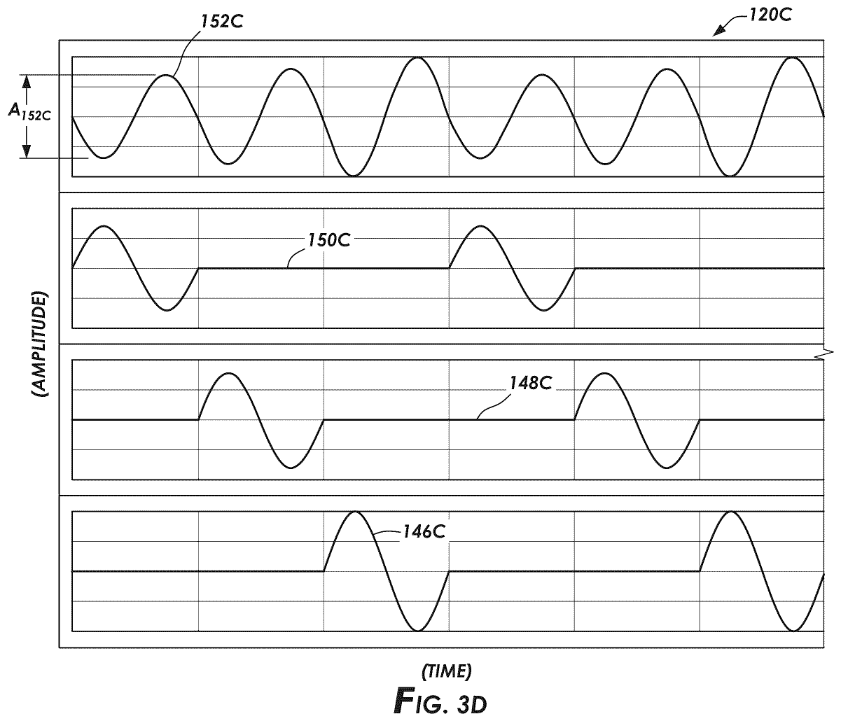

Referring now to FIG. 5, shown in a side sectional schematic view is an example of the low concentration gas cell 44 which includes an entrance 158 (shown in dashed outline) for light to enter within the gas cell 44. Also shown is an exit 160 (in dashed outline) spaced laterally away from the entrance 158 and that allows light to exit, which is then directed towards the photo detector 46 (FIG. 2). In this example, the entrance 158 and exit 160 are each provided on the same axial end of the gas cell 44. Reflectors 162₁₋₃ and 164₁₋₂ are shown inside the gas cell 44 and on opposite ends, with the reflectors 162₁₋₃ on the same end as the entrance 158 and exit 160. Reflectors 162₁₋₃ and 164₁₋₂ along with entrance 158 and exit 160 are shown in a perspective view in FIG. 5A, these are strategically shaped so that light entering through the entrance 158 reflects from and between reflectors 162₁₋₃ and reflectors 164₁₋₂ across the length L₄₄ (FIG. 5) multiple times to increase interaction between light and any analyte gas 71 present inside the low concentration gas cell 44. The configuration of the gas cell 44, which in examples has a length of about 2.5 inches, and due to the multiple reflections has a light path length of around 20 inches (0.5 m), promotes the absorption of light for the detection and identification of the analyte gases 71 present in the sample of ambient fluid 70. Examples exist in which amounts of analyte gases 71 at about 1% on a molar basis are detected and identified and to provide a sub-ppm (0.2 ppm) sensitivity. In embodiments, the low concentration gas cell 44 is the same as or similar to what is referred to as a White cell (https://en.wikipedia.org/wiki/Multipass_spectroscopic_absorption_cells).

The present system allows for field calibration of the device without the use of samples or components exterior of the device for the detection of methane, ethane, carbon monoxide and water as the device contains within all components necessary for calibration. In a non-limiting example of use, calibration is performed with the use of a vial containing known concentrations of one or more gases, such as, methane, carbon dioxide and carbon monoxide. The system can be calibrated with three known calibration points for multiple gases. A first calibration point can be “zero” gas. This occurs with the light beam from a MEMS blackbody infrared light source passing through the high concentration chamber filled with substantially clean atmosphere to the detector and with the light beam from the test and reference sources passing through the low concentration chamber filled with substantially clean atmosphere. A second calibration point is achieved by placing the vial between the MEMS blackbody IR source and the high concentration chamber (filled with clean atmosphere) and passing the light through the vial and high concentration chamber to the detector. A third calibration point is achieved by placing the vial between the test/reference sources and the low concentration chamber (filled with clean atmosphere) and passing the light through the vial and low concentration chamber to the detector.

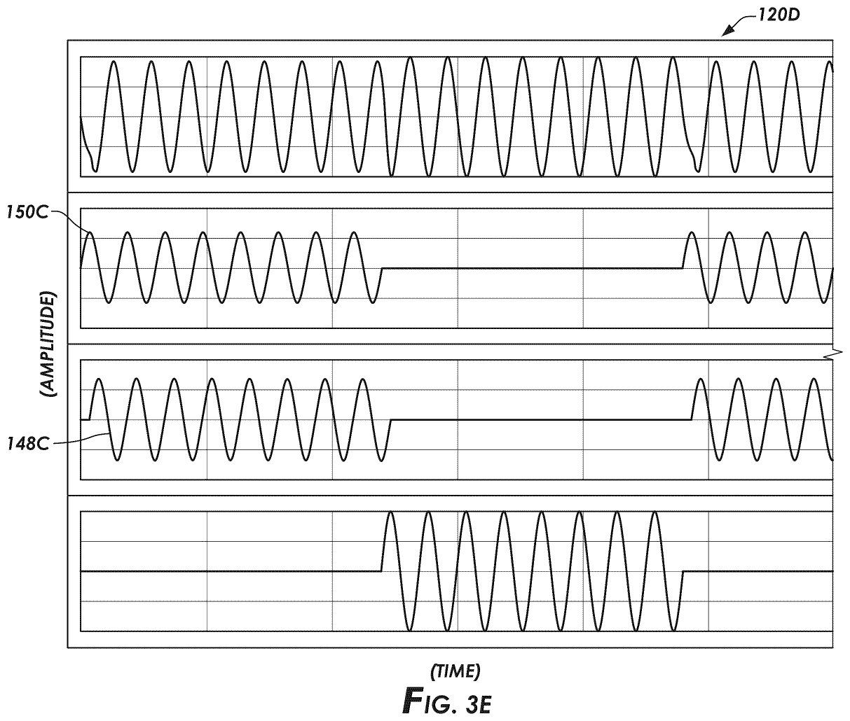

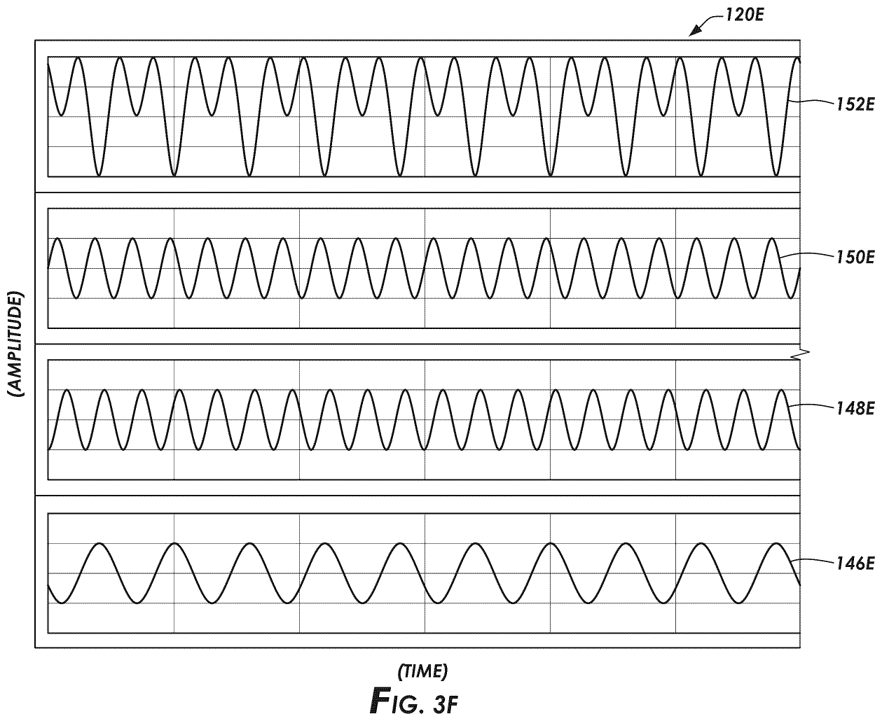

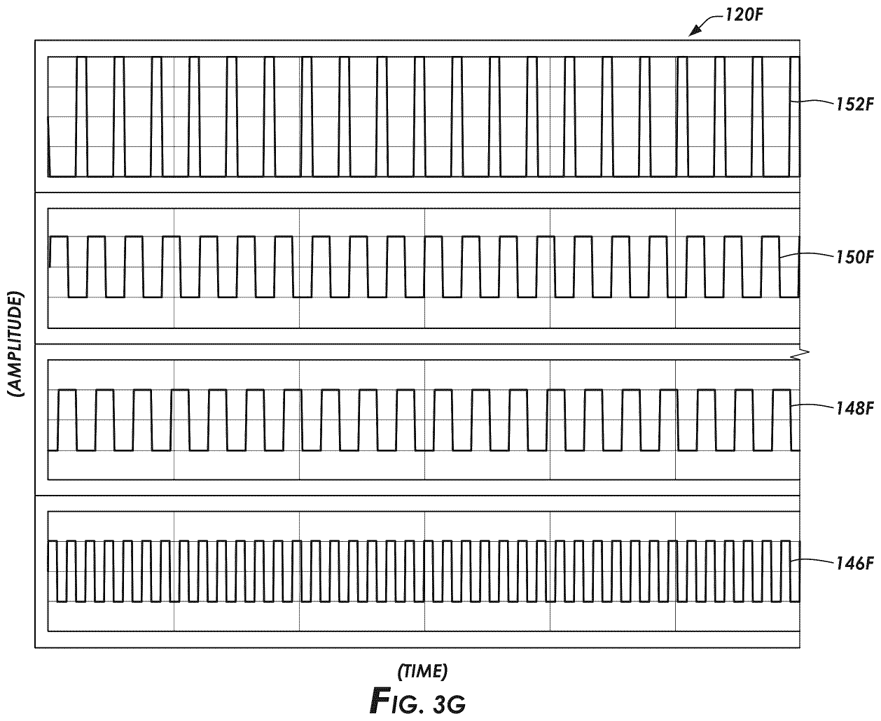

Referring now to FIGS. 6A and 6B, shown in a side elevational view is a schematic example of calibrating the sensing system 10. Included with the sensing system 10 is a calibration system 165 with reference cells or vials 166, 168 that each contain a gas(es) that is(are) in substantially the same concentration in each cell 166, 168. In this example the concentration(s) of the gas(es) is(are) known, and in an embodiment, each cell 166, 168 includes methane, ethane, carbon monoxide, or combinations thereof, and in known concentrations. As shown, the cells 166, 168 are each coupled on ends of arms 170, 172 respectively that are part of a pivoting mechanism. The arms 170, 172 join one another at a pivot point 174 about which the arms 170, 172 and cells 166, 168 are selectively pivoted. In an embodiment, a solenoid (not shown) is also included with the mechanism, which is connected to the mechanism at the pivot point 174 and provides a means for selectively pivoting the arms 170, 172 and cells 166, 168. In the example configuration of FIG. 6A the sensing system 10 is in a “not calibrating” mode in which the cells 166, 168 are both located out of a path of light used for detecting low concentrations of gas, e.g., between splitter 48 (FIG. 1) and cell 46 (“low concentration path”) and also out of a path of light used for detecting high concentrations of gas, e.g., in a path of the light from light source 58 irradiating cell 56 (“high concentration path”). This is the position that the vials are in during normal operation of the device and also the position the cells 166, 168 are in for the first calibration point described above. In order to perform the second and third calibration points described above, the solenoid partially rotates the arms 170, 172 and the cells 166, 168 to a position in which one of the cells 166, 168 intersects the high concentration path and the other vial intersects the low concentration path. In examples, system 10 incorporates a five point calibration by having two cells 166, 168 containing different concentrations of the same gases, and optionally additional or different gases at known concentrations. Examples of the five calibration points include the “zero” gas point as described above. A second calibration point where the first one of the cells 166, 168 intersects the low concentration path; a third calibration point where the second one of the cells 166, 168 intersects the high concentration path; a fourth calibration point where the first one of the cells 166, 168 intersects the high concentration path; and a fifth calibration point where the second one of the cells 166, 168 intersects the low concentration path.

Shown in an overhead view in FIG. 6C is an example of spatial positioning of the calibration system 165 in the housing 68 of the system 10 and with respect to the source assembly 14 and gas cells 44, 56. The pivot point 174 is mounted onto a pivot arm 178 that is supported on a lower wall of housing 68 and pivoted about arm 178 by selective operation of solenoid 176. A non-calibration mode is shown in FIG. 6D, in which arms 170, 172 are oriented about pivot arm 178 so that reference cell 166 is located oblique to cell 56 and cell 168 is located oblique to entrance 158 to cell 44. Locating cells 166, 168 oblique to cell 56 and entrance 158 respectively, positions cell 166 out of the path of light that travels from source 58 to cell 56 and positions cell 168 out of the path of light that travels from lens 43 to entrance 158, which does not interfere with or affect analyte sensing operations disclosed above. An advantage of the present system 10 is the ability to be “field calibrated” at or proximate to the site where analyte testing is being done, and unlike known sensing systems that require a controlled setting, such as a laboratory or certified testing facility. An example of a calibration mode of the system 10 is shown in an elevational view in FIG. 6E illustrating arms 170, 172 being pivoted from the non-calibration mode of FIG. 6D to an orientation so that cell 166 is in the path between lens 43 and entrance 158 and cell 168 is in the path between source 58 and cell 56. In the illustrated example, known concentrations of analyte gas(es) are in each of the reference cells 166, 168 and are the same or substantially the same in each of the cells 166, 168. In a specific example, in each of the cells 166, 168 are known quantities of methane, non-methane hydrocarbons (i.e., ethane, propane, butane, etc.), carbon monoxide, carbon dioxide, oxygen, and combinations thereof. Optionally, prior to the calibration mode cells 44, 56 are purged and refilled with a gas having no or undetectable amounts of analytes detectable by system 10 (i.e., having an absorption band coincident with spectral bands of light generated and/or processed by system 10). An example of a gas in cells 44, 56 during the calibration mode is fresh air drawn into the system 10 at a location spaced away from an area where an analyte(s) is(are) expected or likely to be present. As described above, when in the calibration mode, and while light from lens 43 and/or source 58 irradiates gas in cells 166, 168, signals from detectors 46, 60 provide baseline values relative to known quantities of gas in the cells 166, 168 useful for calibrating system 10.

In a non-limiting example of calibration, the output signal(s) from irradiating the reference cells 166, 168 is(are) compared to an expected signal(s) or signature(s) from a library or database, and software stored in memory (not shown) in the system 10 instructs a processor to correct for any offsets that are outside of a designated tolerance. For example, the system 10 determines the gas concentration by having an internal model of the instrument, which takes the system state (gas concentrations, temperatures, etc.) and outputs expected signals. The system 10 adjusts that input state until the modelled signals match those observed. The model has a series of calibration factors which adjusts output to match a particular instrument, it is the calibration factors that are adjusted by the act of calibration. The adjustments to the calibration factors are made and applied to live software in the system 10 and stored the instrument non-volatile memory. Alternative calibration methods exist, and it is within the capabilities of one skilled in the art to calibrate system 10 based on signal outputs from detectors 46, 58. An advantage of using reference vials with the same gas concentration for calibrating systems having different gas concentration sensitivities, such as the systems described herein having two paths with two gas path lengths (e.g., high concentration of 20 mm and a low concentration of 470 mm) is that the different signals emitted from the different path lengths provides for better accuracy. In an example, obtaining output data from gas cells of different gas path lengths provides knowledge of operation of a detector when detecting an analyte that is in different concentrations in a sample.

In examples, system 10 includes a processor and a non-transitory computer-readable memory accessible by the processor and having executable code (including and in addition to that disclosed above) stored thereon. The executable code includes a set of instructions that causes a processor to perform operations that include controlling operation of LEDs 16, 18, 20, 22 and the light source 58, and adjusting operation of the system 10 and its components based on signals received via communication links 52, 54, 62, 64. Controller/power source 11 optionally includes a computer of any conventional type of suitable processing capacity, such as a personal computer, laptop computer, or any other suitable processing apparatus. It should thus be understood that a number of commercially available data processing systems and types of computers may be used for this purpose.

In a non-limiting example of operation, the system 10 (FIG. 1) is manually transported by a technician or operator, to and/or within an environment where the ambient fluid 70 is to be evaluated for the presence of an analyte gas 71. Electrical power is provided to the system 10 from an electrical source, such as a battery (not shown) coupled to or included with the system 10. The electrical power is directed to the LEDs 16, 18, 20, 22 as described above and via the leads 13 of the bus 12. Resulting signals representing intensity of light sensed by one or more of the photo detectors 46, 60 are transmitted to and received by the controller 11. Data corresponding to the signals is stored in memory within the system 10, or transmitted via hardwire or wireless to a remote location for storage. Based on an analysis of the data, such as by logics and executable code stored in the controller 11 or an associated processor, the presence of an analyte gas 71 in the ambient fluid 70 is detected, and in alternatives an amount of the analyte gas 71 is estimated.

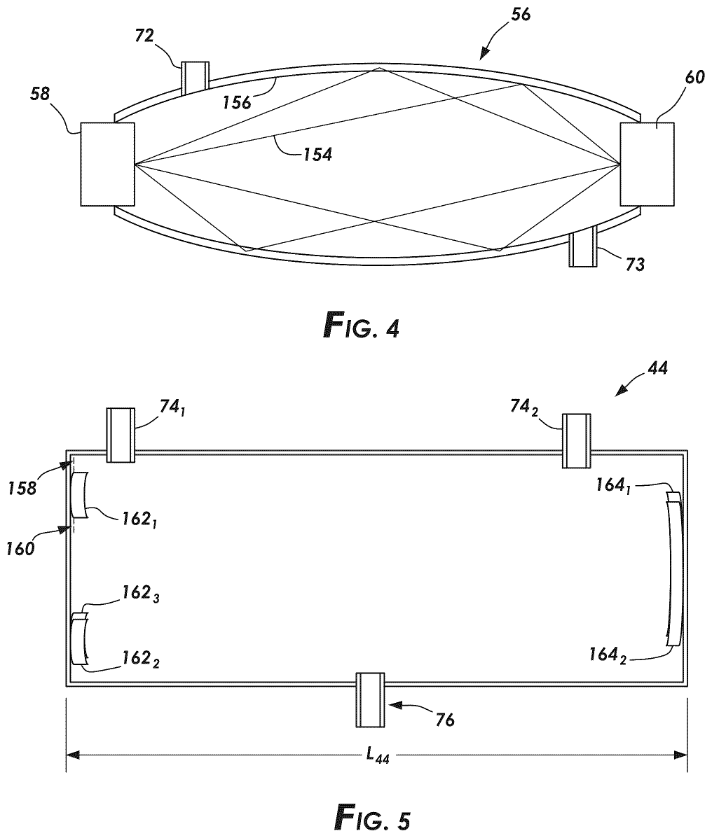

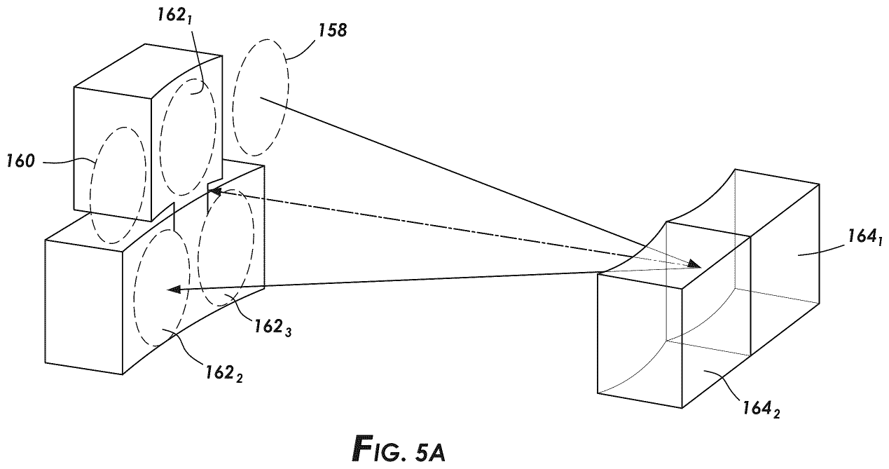

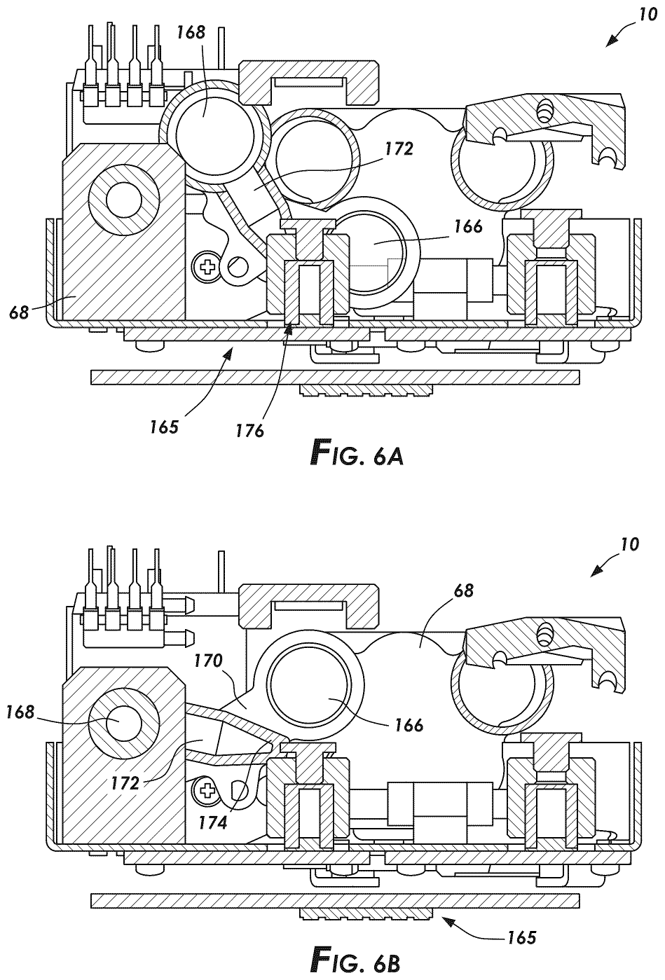

Referring now to FIGS. 7A-7C, shown in a perspective view are example embodiments of multi-gas detection and measurements tools 210, 212, 214, which each include an example of the system 10 of FIG. 1. As described in more detail below, these tools 210, 212, 214 each have a common interface. Tool 210 includes an outer body with an elongated portion, which in embodiments operates as a handle for an operator to hold and operate manually. Tool 210 is referred to herein as a hybrid customer service tool, tool 212 is referred to herein as a hybrid construction tool, and tool 214 is referred to as a hybrid leak survey tool. In alternatives, each of tools 210, 212, and 214 is employed in one or more of leak and odor investigations, pipeline construction pinpointing and barholing, for detecting methane, non-methane hydrocarbons (i.e., ethane, propane, butane, etc.), carbon monoxide, carbon dioxide, and oxygen. Examples of detection sensitivity of these tools 210, 212, 214 are methane at 5 ppm-100%, methane at 0.2 ppm-100%, carbon monoxide at 5 ppm-500 ppm, carbon dioxide at 0.2% to 30%, oxygen at 0.1% to 25%, and non-methane hydrocarbons at 10 ppm-5%. Examples of pipeline gas discrimination include 1% of natural gas mix and alternative, less than 500 ppm of natural gas mix. In examples, tools 210, 212, 214 are self-calibrating, such as the calibration method described above, for gases that include methane, ethane, carbon monoxide, carbon dioxide, and oxygen. Embodiments of tools 210, 212, 214 include removable memory, such as what is referred to as SD cards. Global positioning capabilities are included with the tools 210, 212, 214 via precision dual frequency Global Navigation Satellite System (“GNSS”).

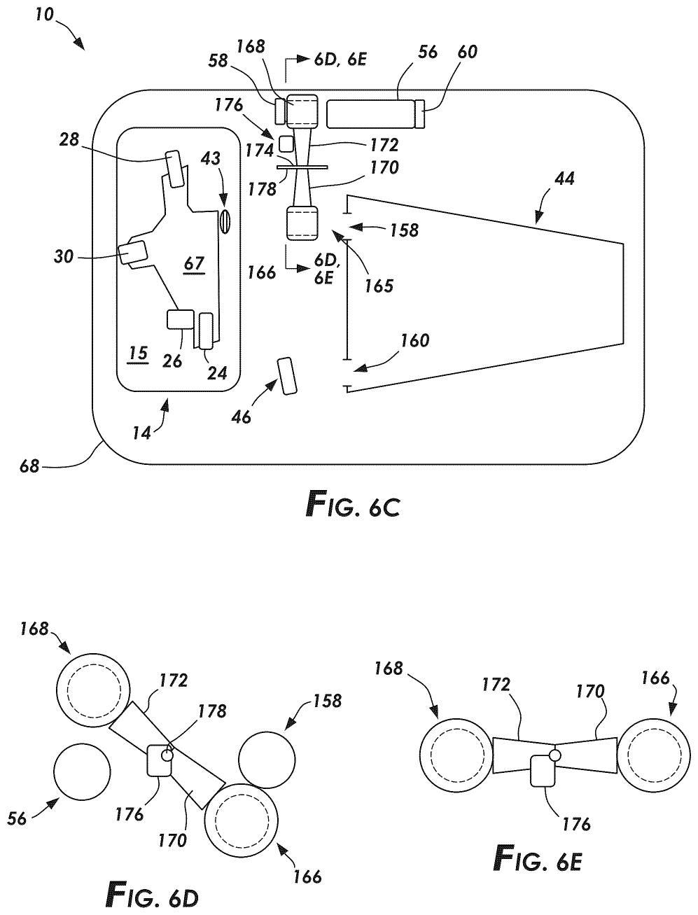

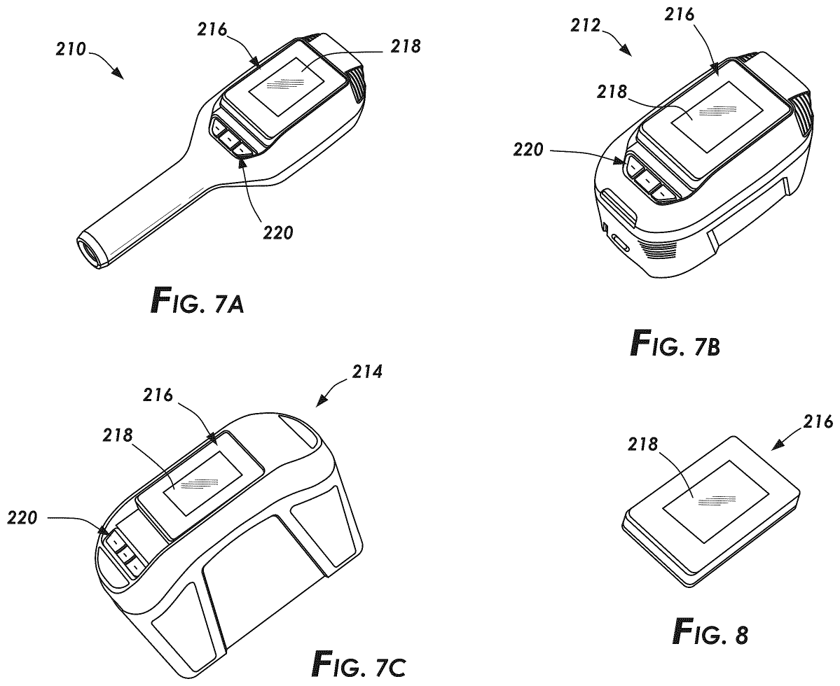

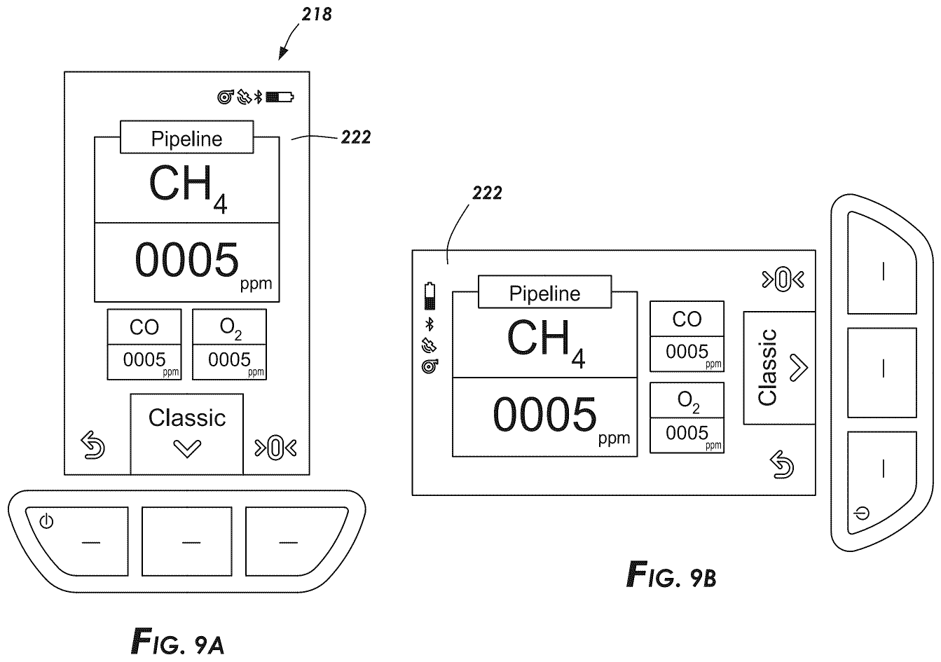

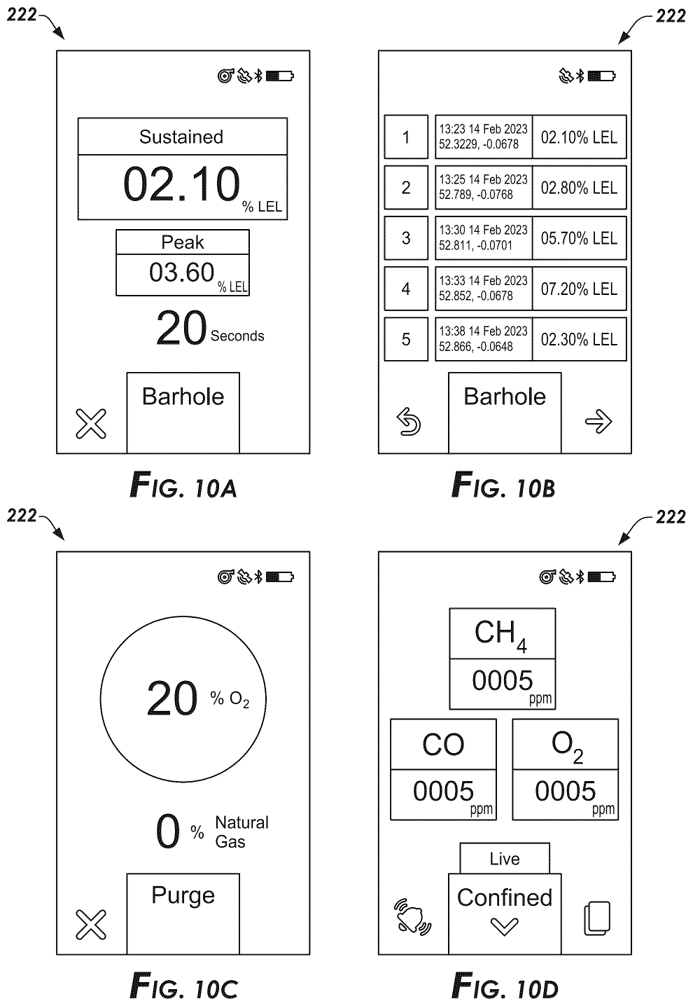

Shown in perspective view in FIG. 8 is an example of a head module 216 that is interchangeable within each of the tools 210, 212, 214 (FIGS. 7A-7C) and that has a common user interface with the tools 210, 212, 214. A screen 218 is shown on the head module 216. Also on the tools 210, 212, 214 is a tactile interface 220 and the screen 218 and the tactile interface 220 optionally operate as a way to input and/or receive output to and from the tools 210, 212, 214. In embodiments, the screen 218 is configured to be a touch screen. Shown in FIGS. 9A and 9B is that on the screen 218 is a visual display 222, which is changeable into either portrait or landscape orientations. Shown in FIGS. 10A-10D are examples of different visual displays presented on the screen 218. An advantage of the head module 216 is that when replaced or changed, data recorded with the tool 210, 212, 214 is alternatively retained within a portion of tool 210, 212, 214 different from the head module 216 and accessible or transferable for processing, or by a replacement head module. In examples, the head module 216 includes a processor, means for wireless communication, user interface software, and alternatively is connectable to a CANBUS protocol connection.

Add on modules (not shown) are optionally included with one or more of the tools 210, 212, 214, examples of which insert into a battery compartment (not shown) provided in each of tools 210, 212, 214. Modules include or connect to a battery (not shown) for powering the particular tool 210, 212, 214 coupled with the module. Examples of functionality of the add on modules includes the ability to analyze ambient fluid 70 (FIG. 1) through means such as thermal conductivity, catalytic, and semi-conductor, so that a greater variety of types of analyte gases are detected. The tools 210, 212, 214 also optionally include add on sensors (not shown) for sensing different types of gas, such as for oxygen, hydrogen, and/or hydrogen sulfide. The tools 210, 212, 214 are also equipped with connections and software for the addition of smart probes, e.g., haptics or quantification.

The present invention described herein, therefore, is well adapted to carry out the objectives and attain the ends and advantages mentioned, as well as others inherent therein. While one or more embodiments have been given for purposes of disclosure, numerous changes exist in the details of procedures for accomplishing the desired results. For example, the modulation scheme(s) disclosed herein are useful for any type of spectroscopy. These are intended to be encompassed within the spirit of the present invention disclosed herein and the scope of the appended claims.

What is claimed is:

1. A method of evaluating an environment comprising:
irradiating a sample volume of the environment with light at a reference wavelength;
irradiating a sample volume of the environment with light at a test wavelength, the test wavelength being a wavelength that is absorbed by an analyte gas and having an intensity that is modulated to be out of phase with an intensity of the light at the reference wavelength;
sensing an intensity of the light at the reference wavelength that is transmitted through the sample volume to define a sensed reference light intensity;
sensing an intensity of the light at the test wavelength that is transmitted through the sample volume to define a sensed test light intensity;
comparing the sensed reference light intensity with the sensed test light intensity; and
identifying the presence of the analyte gas in the environment based on the step of comparing the sensed reference light intensity with the sensed test light intensity.

2. The method of claim 1, wherein the step of comparing the sensed reference light intensity with the sensed test light intensity comprises identifying a change over time of a difference between the sensed reference light intensity and the sensed test light intensity.

3. The method of claim 1, wherein the analyte gas comprises a first analyte gas, the light at the test wavelength comprises a first light, and the test wavelength comprises a first test wavelength, the method further comprising, identifying a second analyte gas present in the sample volume of the environment by irradiating the sample volume of the environment with a second light that is at a second test wavelength, wherein the first light comprises light generated by a first light source and the second light comprises light generated by a second light source having the same characteristics as the first light source.

4. The method of claim 3, further comprising forming the first light by filtering a lower portion of the bandwidth of light from the first light source, and forming the second light by filtering an upper portion of the bandwidth of light from the first light source.

5. The method of claim 4, wherein absorption bands of the first and second analyte gases overlap.

6. The method of claim 1, wherein the light at the test wavelength comprises a first light at the test wavelength and the test wavelength comprises a first test wavelength, the method further comprising irradiating the sample volume of the environment with a plurality of lights at a plurality of test wavelengths, each of the plurality of test wavelengths being different from the first test wavelength and being different from any of the other plurality of test wavelengths, and each of the plurality of lights having an intensity that is modulated to be out of phase with an intensity of the light at the reference wavelength.

7. The method of claim 6, further comprising controlling irradiation of the sample volume so that over a designated period of time the sample volume is irradiated with the first light at the test wavelength or one of the plurality of lights at the plurality of test wavelengths light.

8. The method of claim 7, wherein the sample volume is continuously irradiated with the light at the reference wavelength.

9. The method of claim 6, wherein each of the plurality of test wavelengths being a wavelength that is absorbed by a different analyte gas.

10. The method of claim 1, wherein the environment comprises a space selected from the group consisting of ambient fluid within a residential area, a landfill, a construction site, a gas distribution facility, a gas storage facility, an area where first responders have been summoned, an industrial area, a public area, any area, space or place where the presence and/or quantity of a designated substance is being sensed, detected, or measured, and combinations.

11. The method of claim 1, wherein the analyte gas comprises a gas selected from the group consisting of a noxious gas, carbon monoxide, carbon dioxide, hydrocarbon gases, methane, ethane, and combinations.

12. The method of claim 1, wherein the steps of irradiating and sensing are performed using a sensing system that comprises a controller for identifying the analyte gas and a reference vial having a substance in a known concentration, the method further comprising calibrating the sensing system by irradiating the reference cell with the reference and test lights, sensing the intensity of the test lights being transmitted through the reference cell, and adjusting the sensed test and reference light intensities transmitted through the sample volume based on sensed intensities being transmitted through the reference cell.

13. A system for evaluating an environment comprising:
a reference light source emitting a reference light having a time varying intensity;
a test light source emitting a test light having a time varying intensity modulated to be out of phase with the reference light and having a wavelength that is within an absorption band of an analyte gas;
a space in which a sample volume of the environment is contained;
an optical system comprising, an inlet in the path of the reference light, an inlet in the path of the test light, and an exit;
an emission of light comprising reference light and test light, which projects from the exit and passes through the space and the sample volume of the environment; and
a sensor strategically disposed in a path of the emission of light and on a side of the space opposite the exit.

14. The system of claim 13, wherein the sensor is responsive to an intensity of light, the system further comprising a controller configured to receive signals from the sensor representing light intensity and identify the presence of an analyte gas in the sample volume.

15. The system of claim 13, wherein the optical system comprises dichroic mirrors and collimators for collimating the reference light emitted from the reference light source and the test light from the test light source onto the dichroic mirrors, and wherein the collimators are supported in place by the dichroic mirrors.

16. The system of claim 13, wherein the test light source and test light comprise a first test light source and a first test light, the system further comprising a second test light source having the same characteristics as the first test light source, the optical system comprising a band pass filter having an inlet in the path of the first test light and second test light and an exit from which a modified first test light is emitted having a bandwidth that is in the upper half of a bandwidth of the first test light and a modified second test light is emitted having a bandwidth that is in the lower half of a bandwidth of the second test light, wherein the modified first test light is in the absorption band of a first analyte gas and the modified second test light is in the absorption band of a second analyte gas, and wherein the absorption bands of the first and second analyte gases overlap.

17. The system of claim 13, further comprising a calibration system comprising an arm that is selectively pivotable and a reference vial containing an analyte gas of a known concentration, wherein the reference vial is coupled to the arm and selectively pivoted into the path of the emission of light.

18. The system of claim 13, further comprising a housing covering other elements of the system, a power supply in the housing, and a handle selectively held by an operator when the system is transported to and used in remote environments.

19. The system of claim 13, wherein the sample volume of the environment is contained in a gas cell comprising a container selected from the group consisting of a low concentration gas cell and a high concentration gas cell.

20. The system of claim 13, wherein the test light source and test light comprise a first test light source and a first test light, the system further comprising a plurality of test light sources from which a plurality of test lights are selectively emitted, wherein the first test light and the plurality of test lights are each sequentially emitted and are modulated to be out of phase with the reference light, and wherein the reference light is continuously emitted.
